(19) United States
(12) Patent Application Publication
(10) Pub. No.: US 2009/0142264 A1
(43) Pub. Date: Jun. 4, 2009

(54) 18F-Labeled Phenoxyphenyl Nu-benzyl Alkanamid Derivatives for Positron Emission Tomography (PET) Imaging of Peripheral Benzodiazepine Receptor

(76) Inventors: Bengt Langstrom, Uppsala (SE); Farhad Karimi, Canton, MA (US)

Correspondence Address: GE HEALTHCARE, INC., IP DEPARTMENT, 101 CARNEGIE CENTER, PRINCETON, NJ, 08540-6231, US

(21) Appl. No.: 12/159,016

(22) PCT Filed: Dec. 27, 2006
(86) PCT No.: PCT/IB2006/003775

§ 371 Date: Oct. 27, 2008

Related U.S. Application Data
(60) Provisional application No. 60/754,430, filed on Dec. 28, 2005.

(51) Int. Cl. A61K 51/04; C07C 233/00

(52) U.S. Cl. 424/1.81; 564/123

(57) ABSTRACT

The present invention provides novel ¹⁸F-labeled phenoxyphenyl N-benzyl alkanamid derivative compounds that are suitable for use as an in vivo imaging agent. A pharmaceutical comprising the compound and a kit for the preparation of the pharmaceutical are also provided. Methods of use and use of claims for novel ¹⁸F-labeled phenoxyphenyl N-benzyl alkanamid derivative compounds are provided as well.

### FIELD OF THE INVENTION

The present invention relates to new ¹⁸F-labeled phenoxyphenyl N-benzyl alkanamid derivatives for Positron Emission Tomography (PET). The present invention provides novel ¹⁸F phenoxyphenyl N-benzyl alkanamid derivative compounds that are suitable for use as an in vivo imaging agent. A pharmaceutical comprising the compound and a kit for the preparation of the pharmaceutical are also provided as are methods of use and use of claims for novel ¹⁸F phenoxyphenyl N-benzyl alkanamid derivative compounds that are suitable for use as an in vivo imaging agent.

### BACKGROUND OF THE INVENTION

Tracers labeled with short-lived positron emitting radionuclides (e.g. ¹⁸F, t_{1/2}=110 minutes) are the positron-emitting nuclide of choice for many receptor imaging studies. Accordingly, radiolabeled ligands such as phenoxyphenyl N-benzyl alkanamid derivatives have great clinical potential because of their utility in Positron Emission Tomography (PET) to quantitatively detect and characterize a wide variety of diseases.

The peripheral benzodiazepine receptor (PBR) has been found primarily as a high-affinity binding site for diazepam in rat kidney. Reference. In contrast to the central benzodiazine receptor (CBR), which is associated with gamma-aminobutyic acid_{A} (GABA_{A})-regulated ion channels in the central nervous system, PBR lacks coupling to GABA_{A} receptors.

PBR has been found in many peripheral tissues, in blood cells, and in glial cells in the brain. Its primary localization has been reported to be mainly in the mitochondrial outer membranes in many tissues, although PBR is located on the inner membrane of the rat lung mitochondria. Furthermore, PBR was also found on plasma membranes, which lack mitochondria. Plasma membrane PBR has been described in heart, liver, adrenal, and testis and on hematopoietic cells.

PBR is composed of at least three subunits, an isoquinoline binding subunit with a molecular mass of 18 kDa, a voltage-dependent anion channel (VDAC) with a molecular mass of 32 kDa and an adenine nucleotide carrier with a molecular mass of 30 kDa. cDNA encoding PBR has been cloned from humans, bovines, rats, and mice. PBR plays a role in cell proliferation, steroidogenesis, calcium flow, cellular respiration, cellular immunity, and malignancy. Zhang et al., J. Med. Chem., 2004, vol. 47, pp. 2228-2235.

As endogenous ligands for PBRs the following have been reported: anthraline; diazepam-binding inhibitor (DBI); and proptoporphyrin IV. Anthraline, 16 kDa protein, binds to both PBR and the dihydropyridine binding sites. DBI, a 104 amino acid neuropeptide, has been found in human brain, and DBI-like immunoreactivity has been found in the cerebrospinal fluid of human volunteers. DBI has also been found in peripheral tissues rich in PBRs, such as adrenal glands, testis, and the kidneys. The major physiological porphyrins, protoporphyrin IX and heme, have been labeled PBR with nanomolar affinity, and their affinity has been 1000 times higher for PBRs than for CBRs.

PBR has exhibited different specificities for ligands. Compounds Ro5-4864 and PK11195 as well as imidazopyridine and 2-aryl-3-indoleacetamide derivatives exhibited high affinity for PBRs but not for CBRs.

The physiological functions of PBR have not been fully elucidated, due in part to the lack of potent and selective ligands for PBRs. The pharmacological profile of two high and selective PBR ligands, N-(2,5 Dimethoxylbenzyl)-N-(4-fluoro-2-phenoxyphenyl)acetamide and N-(4-chloro-2-phenoxyphenyl)-N-(2-isopropoxybenzyl)acetamide have been presented. M. Cultry, P. Silver et al., Drug Dev. Research, 2001, vol. 52, 475-484. These compounds ae aryloxyanilide derivatives, and identified with known PBR ligands such as benzodiazepine, isoquinoline, imidazopyridine, and indole derivatives. The aryloxyanilide derivatives, which have been derived by opening a diazepine ring, are a novel class as PBR ligands and have exhibited high and selective affinity for PBRs. Okubo et al, Bioorganic & Medicinal Chemistry, 2004, vol. 12, pp. 423-438. These novel derivatives were used to explore the functions of PBR. Id. The design, synthesis, and structure-affinity relationships of aryloxyanilide derivatives have been described. Id.

Aryloxyanilides have shown promising results as ¹⁸F radioligands for imaging PBRs. ¹⁸F-labeled analogues are advantageous because they are produced in high activity typically 5 GBq, and due to the longer half-life of F-18, the labeled compound can be distributed to other sites for application.

There is a need for further exploring longer fluoroalkyl and branched fluoroalkyl chain acetamide PBR ligands to find a pattern of structure-activity relationships. Studies have been done to identify this pattern by exploring the electron affinity, solvent accessible surface area (SASA), Log P and Log W of known compounds wherein Log P and Log W values show lipophilcity and solubility of the compound. There is also a need to generate novel ¹⁸F aryloxyanilide derivative compounds that possess increased concentration of PBR for PET Imaging to observe in lesioned brain areas in a variety of neutrophathologies and in inflammatory profiles within a patient wherein PBR is a key element of the steroidogenic pathway in peripheral tissues.

The term “analogue” used throughout this invention is defined as a chemical compound that is structurally similar to an acetamide derivative but differs in composition i.e. elements, functional groups. The term “ligand” used throughout this invention is defined as a group, ion, or molecule coordinated to a central atom or molecule in a complex.

Discussion or citation of a reference herein shall not be construed as an admission that such reference is prior art to the present invention.

### SUMMARY OF THE INVENTION

The present invention provides novel ¹⁸F phenoxyphenyl N-benzyl alkanamid derivative compounds that are suitable for use as an in vivo imaging agent. A pharmaceutical comprising the compound and a kit for the preparation of the pharmaceutical are also provided.

The present invention depicts a compound of formula (I)

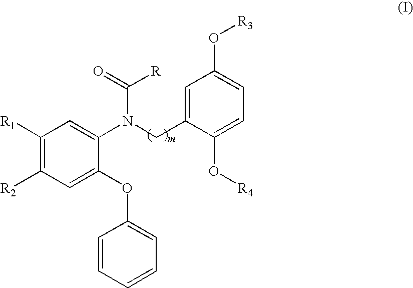

or a salt or solvate thereof, wherein said compound is labeled with an imaging moiety, and wherein,
R=alkyl, aryl, etc
R₁═H, Cl, F, etc
R₂═H, Cl, F, etc
R₃═H, CH₃, F, (CH₂)ₙ—¹⁸F
R₄═H, CH₃, F, (CH₂)ₙ—¹⁸F
n=1C-6C, n-alkyl, branched, deuterated alkyl chain
m=1C-5C

In a further embodiment, the compound of formula (I), wherein

R is alkyl, R₁ is F, R₂ is H or F, R₃ is CH3, R₄ is (CH2)ₙ-¹⁸F, and M is 1C or 2C are also provided.

Yet another embodiment comprises a pharmaceutical composition which comprises the compound of formula (I), wherein the imaging moiety is a radioactive moiety, together with a biocompatible carrier in a form suitable for mammalian administration

In a further embodiment of the present invention comprises a kit of the formula of compound (I),

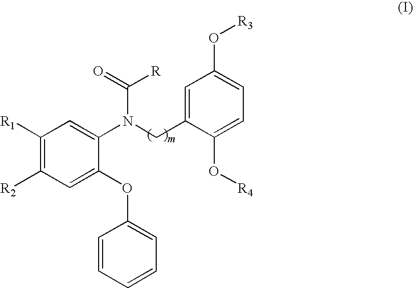

or a salt or solvate thereof, wherein said compound is labeled with an imaging moiety, and wherein,
R=alkyl, aryl, etc
R₁═H, Cl, F, etc
R₂═H, Cl, F, etc
R₃═H, CH₃, F, (CH₂)ₙ—¹⁸F
R₄═H, CH₃, F, (CH₂)ₙ—¹⁸F
n=1C-6C, n-alkyl, branched, deuterated alkyl chain
m=1C-5C
   further wherein said kit is suitable for the preparation of a pharmaceutical composition according to claim 6.

Yet in another embodiment of the invention, a method for the in vivo diagnosis or imaging of a PBR-related condition in a subject is claimed that comprises administration of a pharmaceutical composition comprising a compound of claim 8.

The present invention also provides a method of monitoring the effect of treatment of a human or animal body with a drug to combat a PBR-related condition, said method comprising administering to said body the pharmaceutical composition of claim 6, and detecting the uptake of said pharmaceutical.

### DETAILED DESCRIPTION OF THE INVENTION

In the current invention, ¹⁸F-labeled analogues were developed based on aryloxynilides. Aryloxyanilides have showed promising results as radioligands for imaging peripheral type benzodiazepine binding site (PBR). Efficient ¹⁸F-labeled analogues such as phenoxyphenyl N-benzyl alkanamid derivative compounds have special value, since they can be produced in high activity and distributed to other nearby sites for application.

After obtaining the ¹⁸F-labeled phenoxyphenyl N-benzyl alkanamid derivative compounds, using an automated system termed FastLab or Tracerlab, high performance liquid chromatography (HPLC) is used to verify the structure of the analogues. A further tool was used to verify the structure of the analogues wherein a calculation study was conducted to look into the physical properties and 3D images of various analogues. The calculation study was conducted using a computer-aided molecular design modeling tool also know as CAChe. CAChe enables one to draw and model molecules as well as perform calculations on a molecule to discover molecular properties and energy values. The calculations are performed by computational applications, which apply equations from classical mechanics and quantum mechanics to a molecule. For example, the claimed novel compounds such as the ¹⁸F-labeled phenoxyphenyl N-benzyl alkanamid derivative compounds were designed using CAChe.

There are several advantages for using the claimed compounds for (Positron Emission Tomography) PET Imaging of PBRs. Inherent advantages are the high affinity of these compounds toward PBR wherein the high affinity is reached using longer fluoroalkyl and branched chains. Another advantage of using the claimed compounds are their structure-activity relationships with PBR.

Below a detailed description is given of ¹⁸F phenoxyphenyl N-benzyl alkanamid derivative compounds that are suitable for use as an in vivo imaging agent. A pharmaceutical comprising the compound and a kit for the preparation of the pharmaceutical are also provided.

In one embodiment of the present invention comprises a compound of formula (I),

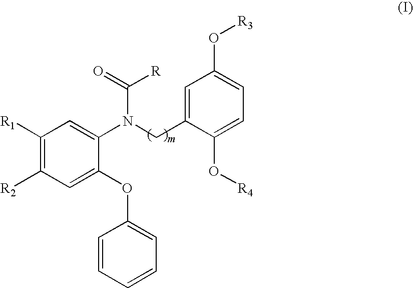

or a salt or solvate thereof, wherein said compound is labeled with an imaging moiety, and wherein,
R=alkyl, aryl, etc
R₁═H, Cl, F, etc
R₂═H, Cl, F, etc
R₃═H, CH₃, F, (CH₂)ₙ—¹⁸F
R₄═H, CH₃, F, (CH₂)ₙ—¹⁸F
n=1C-6C, n-alkyl, branched, deuterated alkyl chain
m=1C-5C

Another embodiment of the present invention comprises a compound according to formula (I), wherein R is alkyl, R₁ is F, R₂ is H or F, R₃ is CH₃, R₄ is (CH2)ₙ-¹⁸F, and M is 1C or 2C.

Yet a further embodiment of the present invention comprises a compound of formula (I), wherein said imaging moeity comprises a positron-emitting radioactive non-metal.

A further embodiment comprises a compound of formula (I), wherein said imaging moeity is a positron-emitting radioactive non-metal selected from the group consisting of ¹¹C and ¹⁸F.

An additional embodiment includes the compound of formula (I), wherein said positron-emitting radioactive non-metal is ¹⁸F.

Yet a further embodiment includes a pharmaceutical composition which comprises the compound of formula (I), wherein the imaging moiety is a radioactive moiety, together with a biocompatible carrier in a form suitable for mammalian administration.

In another embodiment of the present invention, the pharmaceutical composition of formula (I), wherein the pharmaceutical composition is a radiopharmaceutical is also provided.

A further embodiment includes a kit comprising the formula of compound (I),

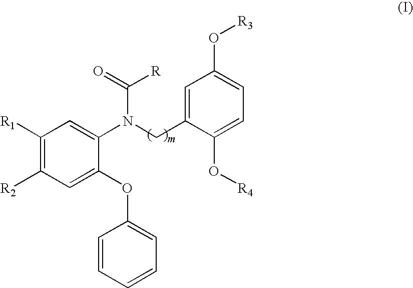

or a salt or solvate thereof, wherein said compound is labeled with an imaging moiety, and wherein,
R=alkyl, aryl, etc
R₁═H, Cl, F, etc
R₂═H, Cl, F, etc
R₃═H, CH₃, F, (CH₂)ₙ—¹⁸F
R₄═H, CH₃, F, (CH₂)ₙ—¹⁸F
n=1C-6C, n-alkyl, branched, deuterated alkyl chain
m=1C-5C

further wherein said kit is suitable for the preparation of a pharmaceutical composition wherein the imaging moiety is a radioactive moiety, together with a biocompatible carrier in a form suitable for mammalian administration.

The kits comprise a suitable precursor of the second embodiment, preferably in sterile non-pyrogenic form, so that reaction with a sterile source of an imaging moiety gives the desired pharmaceutical with the minimum number of manipulations. Such considerations are particularly important for radiopharmaceuticals, in particular where the radioisotope has a relatively short half-life, and for ease of handling and hence reduced radiation dose for the radiopharmacist. Hence, the reaction medium for reconstitution of such kits is preferably a “biocompatible carrier” as defined above, and is most preferably aqueous.

Suitable kit containers comprise a sealed container which permits maintenance of sterile integrity and/or radioactive safety, plus optionally an inert headspace gas (e.g. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

The kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation. The “biocompatible cation” and preferred embodiments thereof are as described above. By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the radioactive imaging product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the conjugate is employed in acid salt form, the pH adjusting agent may optionally be provided in a separate vial or container, so that the user of the kit can adjust the pH as part of a multi-step procedure.

The term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The “biocompatible carrier” is a fluid, especially a liquid, in which the compound is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Furthermore, the pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. For radiopharmaceutical compositions, the pre-filled syringe may optionally be provided with a syringe shield to protect the operator from radioactive dose. Suitable such radiopharmaceutical syringe shields are known in the art and preferably comprise either lead or tungsten. The radiopharmaceuticals may be administered to patients for SPECT or PET imaging in amounts sufficient to yield the desired signal, typical radionuclide dosages of 0.01 to 100 mCi, preferably 0.1 to 50 mCi will normally be sufficient per 70 kg bodyweight.

Another embodiment comprises a method for the in vivo diagnosis or imaging of a PBR-related condition in a subject, further comprising administration of a pharmaceutical composition comprising a compound of formula (I).

An in vivo diagnostic or imaging method, e.g. SPECT or PET relates to the in vivo imaging of PBR and therefore has utility in the diagnosis of PBR-related conditions. Examples of PBR-related conditions include malignancy, and neuropathologies such as multiple sclerosis, Alzheimer's disease and Huntington's disease.

The present invention also provides a method of monitoring the effect of treatment of a human or animal body with a drug to combat a PBR-related condition, said method comprising administering to said body the pharmaceutical composition of claim 6, and detecting the uptake of said pharmaceutical.

The present invention further provides a precursor for the preparation of the compound of formula (I) wherein said precursor is derivatized to include a chemical group suitable for labeling with an imaging moiety.

Another embodiment of the present invention, is that the chemical group of the precursor of formula (I) is suitable for labeling with a radioactive imaging moiety.

A precursor comprises a derivative of the compound of Formula I, designed so that chemical reaction with a convenient chemical form of the imaging moiety occurs site-specifically; can be conducted in the minimum number of steps; and without the need for significant purification, to give the desired imaging agent. Such precursors are synthetic and can conveniently be obtained in good chemical purity. The “precursor” may optionally comprise a protecting group for certain functional groups of the compound of Formula I.

An additional embodiment of the invention is the use of a compound of formula (I),

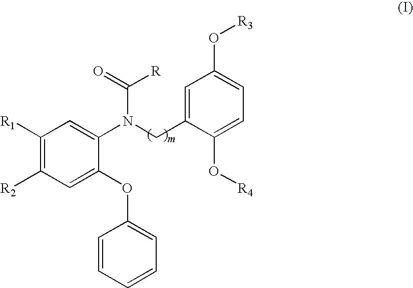

or a salt or solvate thereof, wherein said compound is labeled with an imaging moiety, and wherein,
R=alkyl, aryl, etc
R₁═H, Cl, F, etc
R₂═H, Cl, F, etc
R₃═H, CH₃, F, (CH₂)ₙ—¹⁸F
R₄═H, CH₃, F, (CH₂)ₙ—¹⁸F
n=1C-6C, n-alkyl, branched, deuterated alkyl chain
m=1C-5C

Yet another further embodiment of the present invention claims a method of use for monitoring the effect of treatment of a human or animal body with a drug to combat a PBR-related condition, said method comprising

administering to said body the pharmaceutical composition of claim 6, and detecting the uptake of said pharmaceutical.

Still a further embodiment of the present invention encompasses the use of a precursor for the preparation of the compound of claim 1 wherein said precursor is a compound of Formula (I) derivatized to include a chemical group suitable for labeling with an imaging moiety.

Another embodiment of the present invention describes the use of a kit comprising the formula of compound (I),

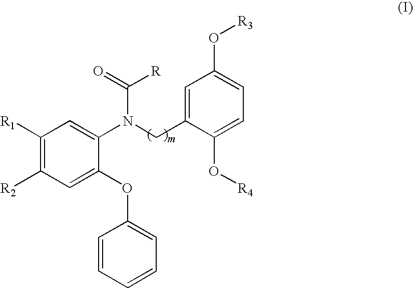

or a salt or solvate thereof, wherein said compound is labeled with an imaging moiety, and wherein,
R=alkyl, aryl, etc
R₁═H, Cl, F, etc
R₂═H, Cl, F, etc
R₃═H, CH₃, F, (CH₂)ₙ—¹⁸F
R₄═H, CH₃, F, (CH₂)ₙ—¹⁸F
n=1C-6C, n-alkyl, branched, deuterated alkyl chain
m=1C-5C
and further wherein said kit is suitable for the preparation of a pharmaceutical composition according to claim 6.

The term “protecting group” means a group which inhibits or suppresses undesirable chemical reactions, but which is designed to be sufficiently reactive that it may be cleaved from the functional group in question under mild enough conditions that do not modify the rest of the molecule. After deprotection the desired product is obtained.

The term “metal complex” means a coordination complex of the metal ion with one or more ligands. It is strongly preferred that the metal complex is “resistant to transchelation”, i.e. does not readily undergo ligand exchange with other potentially competing ligands for the metal coordination sites.

### EXAMPLES

The invention is further described in the following examples which are in no way intended to limit the scope of the invention.

Experimental Studies

General Method for Preparing ¹⁸F-Labeled Novel ¹⁸F-Labeled Phenoxyphenyl N-benzyl Alkanamid Derivative Compounds

A solution of the corresponding precursor in proper anhydrous solvent was added to dry the [K/K2.2.2]⁺¹⁸F⁻. The reaction mixture was heated at 150° C. for 15 minutes. The crude mixture was analyzed and purified by analytical High Performance Liquid Chromotography (HPLC).

Preparation of the [K/K2.2.2]⁺¹⁸F⁻ (Using Enriched 95% ¹⁸O Water)

After irradiation, the target content was passed through a pre-conditioned QMA cartridge resin. The column was purged with helium for five minutes. The [¹⁸F]fluoride adsorbed on the resin was eluted into a reaction vial with 4 ml of a 96:4 (by volume) acetonitrile-water mixture containing 19.1 mg of kryptofix 2.2.2, wherein kryptofix 2.2.2 is a base transfer catalyst that transports the ¹⁸F-fluoride into the organic phase where the reaction take place, and 2.9 mg of K₂CO₃; the solution was then evaporated and co-evaporated with anhydrous acetonitrile (2×1 ml) to dryness in a nitrogen stream at 110° C. as shown below.

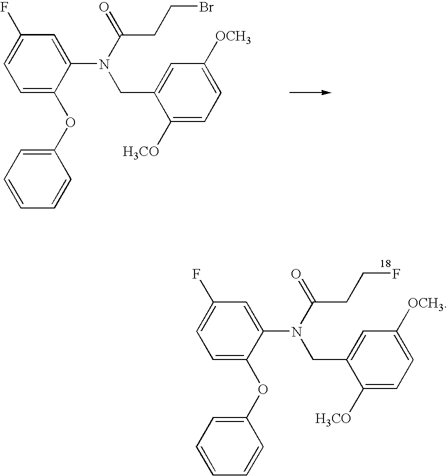

Synthesis of ¹⁸F-Labeled Novel ¹⁸F-Labeled Phenoxyphenyl N-benzyl Alkanamid Derivative Compounds

¹⁸F-labeled novel ¹⁸F-labeled phenoxyphenyl N-benzyl alkanamid derivative compounds could be retro-synthesized as follows:

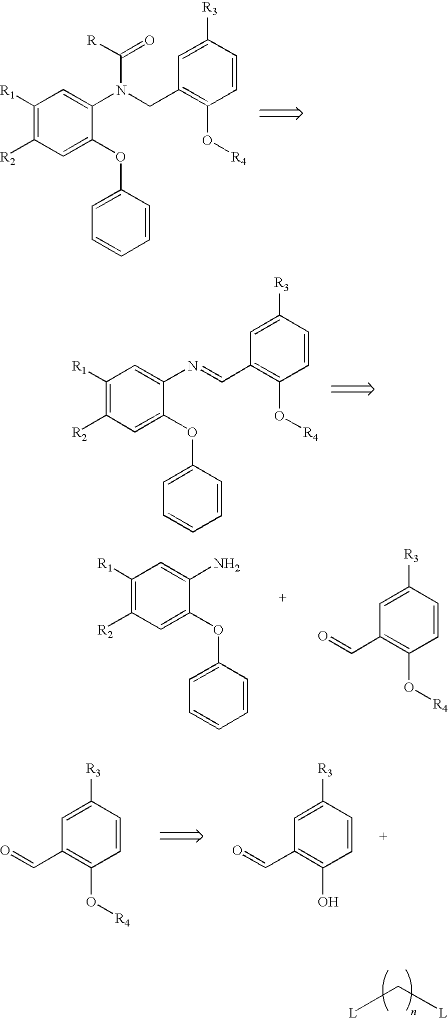

wherein R1 is H, Cl, F, or the a similar halogen; R₂ is H, Cl, F, or a similar halogen; R₃ is H, CH₃, F, (CH₂)n-¹⁸F; R₄ is H, CH₃, F, (CH₂)n-¹⁸F; and L could be any proper leaving group such as Br, I, Cl, TsO, MsO, R_{f}SO₃.

### SPECIFIC EMBODIMENTS, CITATION OF REFERENCES

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

1. A compound of formula (I), 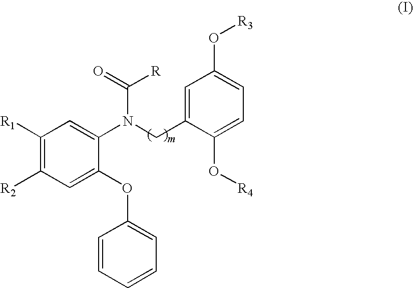
or a salt or solvate thereof, wherein said compound is labeled with an imaging moiety, and wherein,
R=alkyl, aryl, etc
R₁═H, Cl, F, etc
R₂═H, Cl, F, etc
R₃═H, CH₃, F, (CH₂)ₙ—¹⁸F
R₄═H, CH₃, F, (CH₂)ₙ—¹⁸F
n=1C-6C, n-alkyl, branched, deuterated alkyl chain
m=1C-5C

2. The compound according to claim 1, wherein
R is alkyl, R₁ is F,
R₂ is H or F,
R₃ is CH3,
R₄ is (CH2)ₙ-¹⁸F, and
M is 1C or 2C.

3. The compound according to claim 1, wherein said imaging moeity comprises a positron-emitting radioactive non-metal.

4. The compound according to claim 3, wherein said imaging moeity is a positron-emitting radioactive non-metal selected from the group consisting of 11C and 18F.

5. The compound according to claim 4, wherein said positron-emitting radioactive non-metal is 18F.

6. A pharmaceutical composition which comprises the compound of claim 1, wherein the imaging moiety is a radioactive moiety, together with a biocompatible carrier in a form suitable for mammalian administration.

7. The pharmaceutical composition according to claim 6, wherein the pharmaceutical composition is a radiopharmaceutical.

8. A kit comprising the formula of compound (I), 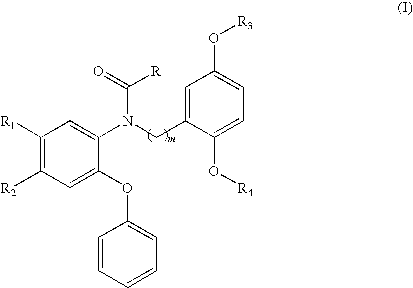
or a salt or solvate thereof, wherein said compound is labeled with an imaging moiety, and wherein,
R=alkyl, aryl, etc
R₁═H, Cl, F, etc
R₂═H, Cl, F, etc
R₃═H, CH₃, F, (CH₂)ₙ—¹⁸F
R₄═H, CH₃, F, (CH₂)ₙ—¹⁸F
n=1C-6C, n-alkyl, branched, deuterated alkyl chain
m=1C-5C
further wherein said kit is suitable for the preparation of a pharmaceutical composition according to claim 6.

9. A method for the in vivo diagnosis or imaging of a PBR-related condition in a subject, comprising administration of a pharmaceutical composition comprising a compound of claim 8.

10. A method of monitoring the effect of treatment of a human or animal body
with a drug to combat a PBR-related condition, said method comprising administering to said body the pharmaceutical composition of claim 6, and detecting the uptake of said pharmaceutical.

11. A precursor for the preparation of the compound of claim 1 wherein said
precursor is a compound of Formula (I) derivatized to include a chemical group suitable for labeling with an imaging moiety.

12. The precursor of claim 11 wherein said chemical group is suitable for labeling
with a radioactive imaging moiety.

13. Use of a compound of formula (I), 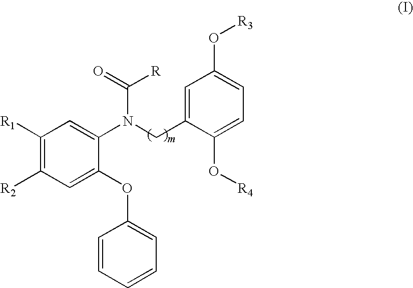
or a salt or solvate thereof, wherein said compound is labeled with an imaging moiety, and wherein,
R=alkyl, aryl, etc
R₁═H, Cl, F, etc
R₂═H, Cl, F, etc
R₃═H, CH₃, F, (CH₂)ₙ—¹⁸F
R₄═H, CH₃, F, (CH₂)ₙ—¹⁸F
n=1C-6C, n-alkyl, branched, deuterated alkyl chain
m=1C-5C

14. A compound comprising formula (I), wherein said compound is further defined in claim 7.

15. The method of use for monitoring the effect of treatment of a human or animal body with a drug to combat a PBR-related condition, said method comprising administering to said body the pharmaceutical composition of claim 6, and detecting the uptake of said pharmaceutical.

16. The use of a precursor for the preparation of the compound of claim 1 wherein said precursor is a compound of Formula (I) derivatized to include a chemical group suitable for labeling with an imaging moiety.

17. The use of a kit comprising the formula of compound (I), 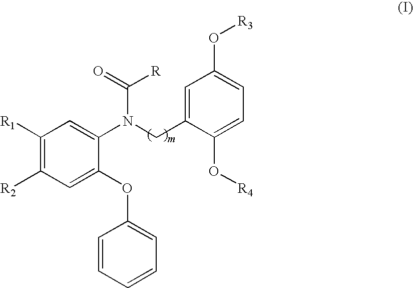
or a salt or solvate thereof, wherein said compound is labeled with an imaging moiety, and wherein,
R=alkyl, aryl, etc
R₁═H, Cl, F, etc
R₂═H, Cl, F, etc
R₃═H, CH₃, F, (CH₂)ₙ—¹⁸F
R₄═H, CH₃, F, (CH₂)ₙ—¹⁸F
n=1C-6C, n-alkyl, branched, deuterated alkyl chain
m=1C-5C
and further wherein said kit is suitable for the preparation of a pharmaceutical composition according to claim 6.
